(19) United States
(12) Patent Application Publication
(10) Pub. No.: US 2011/0236430 A1
(43) Pub. Date: Sep. 29, 2011

(54) METHOD FOR CONTROLLING TOXICITY OF METALLIC PARTICLE AND LOW-TOXICITY COMPOSITE OF METALLIC NANOPARTICLE AND INORGANIC CLAY

(75) Inventors: Jiang-Jen Lin, Taipei (TW); Hong-Lin Su, Taipei (TW); Fu-Chuo Peng, Taipei (TW); Chia-Yu Chu, Taipei (TW); Ying-Fang Chiu, Taipei (TW); Pei-Ru Li, Taipei (TW); Hsi-Tai Sun, Taipei (TW)

(73) Assignee: NATIONAL TAIWAN UNIVERSITY, Taipei (TW)

(21) Appl. No.: 13/012,767

(22) Filed: Jan. 24, 2011

(30) Foreign Application Priority Data
Mar. 26, 2010 (TW) 099109262

(51) Int. Cl. A61K 33/24; A61K 9/00; A61K 33/00; A61K 33/38; A61K 33/34; A61K 33/26; A61P 17/02; A61P 31/00; B82Y 5/00

(52) U.S. Cl. 424/400; 424/600; 424/649; 424/618; 424/630; 424/646; 977/915

(57) ABSTRACT

The present invention provides a method for controlling toxicity of metallic particles and a low-toxicity composite of metallic nanoparticles and inorganic clay. The metallic nanoparticles are effective in preventing infection and in skinning over, and thus suitable for treating scalds/burns. In the composite, the weight ratio of metallic nanoparticles to inorganic clay preferably ranges 0.1/99.9 to 6.0/94.0 in a size of about 5 to 100 nm. Preferably, the metal is silver and the inorganic clay is nano silicate platelets.

### BACKGROUND OF THE INVENTION

1. Field of the Invention

The present invention relates to a composite of metallic particles and clay, and particularly to a low-toxicity composite of metallic nanoparticles and inorganic clay. The present invention also relates to a method for controlling the toxicity of metallic particles, and particularly to a method for controlling the toxicity of metallic particles by complexing the metallic particles with inorganic clay. The present invention can be applied to pharmaceuticals for preventing infection and treating scalds/burns.

2. Related Prior Art

Silver is known as an effective component for antibacterial purpose and for treating wounds. However, its cytotoxicity and genotoxicity should be considered.

So far, silver sulfadiazine is effective in treating scalds/burns due to its wide effects in killing Gram positive bacteria, Gram negative bacteria and fungi. However, sulfadiazine pharmaceuticals can cause side effects, for example, hepatitis, anemia from bone marrow suppression, crystalluria, and lesions of neural and gastrointestinal system.

On the contrary, silver nanoparticles have low cell stimulating and cytotoxicity to human bodies and long-term and strong antibacterial effect, and therefore are suitable for replacing silver sulfadiazine. For metals, inorganic layered clay and exfoliated nanosilicate platelets (NSP) are good dispersants, carriers and protectors. Accordingly, the present invention attempts to complex inorganic layered clay and nanosilicate platelets with silver nanoparticles to improve pharmaceuticals containing silver.

### SUMMARY OF THE INVENTION

An object of the present invention is to provide a method for controlling the toxicity of metallic nanoparticles, so that the metallic nanoparticles can be used to treat scalds/burns and enhance skinning over without infection.

Another object of the present invention is to provide a low-toxicity composite of metallic nanoparticles and inorganic clay, so that the composite can be used as one of pharmaceutical components for treating scalds/burns.

In the present invention, the method for controlling the toxicity of metallic particles is to mix the metallic nanoparticles, layered inorganic clay and a reducing agent to form a composite of the metallic nanoparticles and the inorganic clay. The composite has a size from 5 nm to 100 nm and the weight ratio of the metallic nanoparticles to the layered inorganic clay ranges from 0.1/99.9 to 6.0/94.0.

The layered inorganic clay has an aspect ratio about 10 to 100,000 and serves as carriers of the metallic nanoparticles so that the metallic nanoparticles can be dispersed on a nano scale. The reducing agent can be methanol, ethanol, propanol, butanol, formaldehyde, ethylene glycol, propylene glycol, butanediol, glycerine, PVA (polyvinyl alcohol), PEG (polyethylene glycol), PPG (polypropylene glycol), dodecanol or sodium borohydride (NaBH₄). The reaction is preferably performed with ultrasonic mixing at 25° C. to 100° C. for 1 hour to 20 hours.

In the present invention, the metal can be gold, silver, copper or iron; and silver is preferred. The layered inorganic clay can be nanosilicate platelets (NSP), montmorillonite (MMT), bentonite, laponite, synthetic mica, kaolinite, talc, attapulgite clay, vermiculite or layered double hydroxides (LDH); and the NSP is preferred. The weight ratio of the metallic nanoparticles to the layered inorganic clay preferably ranges from 0.5/99.5 to 3.0/97.0, and more preferably from 0.5/99.5 to 2.0/98.0. The layered inorganic clay preferably has an aspect ratio ranging from 100 to 1,000 and cation exchange equivalent ranging from 0.1 mequiv/g to 5.0 mequiv/g.

The composite of the metallic nanoparticles and the inorganic clay can be used to produce pharmaceuticals for inhibiting growth of bacteria on a chronic wound or enhancing skinning over of a peracute wound.

In a preferred embodiment of the present invention, silver nanoparticles (AgNPs) and NSP form a AgNP/NSP composite. Each AgNP (about 25 nm) includes about 250 silver atoms, and each NSP can complex with about six to eight AgNPs on the surface thereof. When the concentration of the AgNP/NSP composite is 0.01 to 0.05 wt %, the skin-infective bacteria can be completely inhibitted, for example, Candida albicans, pseudomonas aeruginosa, staphylococcus aureus, streptococcus pyogenes and proteus. For meticillin-resistant staphylococcus aureus (MRSA) and fungi, the AgNP/NSP composite is also effective.

### BRIEF DESCRIPTION OF THE DRAWINGS

FIGS. 1˜5 show the effects of the AgNP/NSP composite in inhibiting the growth of five kinds of skin-infective bacteria.

FIGS. 6˜7 show the results of the in vitro cytotoxicity tests of the AgNP/NSP composite on mammals.

FIGS. 8˜10 show the results of the in vitro cytotoxicity tests of the AgNP/NSP composite at different weight ratios on mammals.

FIG. 11 shows the in vitro genotoxicity test of the AgNP/NSP composite on mammals.

FIG. 12 shows the effects of the AgNP/NSP composite in skinning over of peracute scalds/burns.

FIG. 13 shows the effects of the AgNP/NSP composite in skinning over of chronic knife wounds.

### ATTACHMENTS

ATTACHMENT 1 shows the gene mutation assay of the bacteria without enzyme metabolism (−S9).

ATTACHMENT 2 shows the gene mutation assay of the bacteria with enzyme metabolism (+S9).

### DETAILED DESCRIPTION OF THE PREFERRED EMBODIMENTS

The materials used in the preferred embodiments and applications of the present invention include:
1. Nanosilicate platelets (NSP): available by exfoliating montmorillonite (Na⁺-MMT), as described in U.S. Pat. No. 7,125,916, U.S. Pat. No. 7,094,815, and U.S. Pat. No. 7,022,299 or Publication Nos. US 2006-0287413-A1 and US 2006-0063876A1.
2. AgNO₃: used for exchanging or replacing Na⁺ between layers of the inorganic clay to be reduced to Ag nanoparticles.
3. NaBH₄: a strong reducing agent for silver ions.
4. Methanol: CH₃OH, 95%, a weak reducing agent, used to reduce the silver ions into silver nanoparticles at 30˜150° C.
5. Ethylene glycol: C₂H₄(OH)₂, a weak reducing agent, used to reduce the silver ions into silver nanoparticles at 30˜150° C.
6. Silver sulfadiazine: produced by Sinphar Pharmaceutical Co., Ltd., trade mark name Silvazine®, including silver 2.6 mM, equal to 0.5 wt % of AgNP/SWN.
7. Aquacel: pharmaceutical dressing including silver, produced by Bristol-Myers Squibb Company.
8. Microorganism:
(1) staphylococcus aureus (71, 431 and 10781 strains), streptococcus pyogenes (Rob 193-2 strain), pseudomonas aeruginosa, salmonella (4650 and 4653 strains) and Escherichia: coli isolated from wild colonies and provided by Dr. Lin Chun-Hung of Animal Technology Institute Taiwan.
(2) Preparation of standard suspensions of bacteria
   The suspensions of bacteria cultured overnight were added into a fresh Luria-Bertani (LB) liquid media at a volume ratio of 1/100 to be cultured for about three hours. Absorbance (OD₆₀₀) of the suspensions of bacteria after culturing was determined with a spectrophotometer, and the suspensions having OD₆₀₀ values ranging between 0.4 to 0.6 were selected as the standard suspensions of bacteria.

In the present invention, the preferred natural and synthetic clay includes:
1. Bentonite: layered silicate clay having cationic exchange capacity (CEC)=0.67 mequiv/g, purchased from CO—OP Chemical Co., trademark name SWN.
2. Synthetic fluorine mica: product of CO—OP Chemical Co. (Japan), code number SOMASIF ME-100, with cationic exchange capacity (CEC)=1.20 mequiv/g.
3. Layered silicate clay: Laponite, product of The Far Eastern Trading Co., LTD., with cationic exchange capacity (CEC)=0.69 mequiv/g.
4. Synthetic layered double hydroxide:
   [^{II}₁₋ₓM^{III}ₓ(OH)₂]ᵢₙₜᵣₐ[Aⁿ⁻·nH₂O]ᵢₙₜᵣₐ wherein M^{II} is Mg, Ni, Cu or Zn; M^{III} is Al, Cr, Fe, V or Ga; Aⁿ⁻ is Co₃²⁻ or No₃⁻; with ionic exchange capacity in the range of 2.0 to 4.0 mequiv./g.

The low-toxicity AgNP/NSP composite of the present invention can be tested as follows to verify effects thereof.

### A. Inhibition of Growth of Bacteria in Liquid Media Including the AgNP/NSP Composites

The AgNP/NSP composites in different concentrations were prepared respectively in 10 ml LB liquid media, and then five kinds of bacteria (Candida albicans, streptococcus pyogenes, staphylococcus aureus, proteus and pseudomonas aeruginosa) were respectively added to form 100λ standard suspensions. After being cultured at 37° C. for 3 and 24 hours, each suspension was sampled and diluted. 50λ of each dilution was spread on a 10 mm solid LB medium with a sterilized glass bead and cultured at 37° C. for 24 hours. The numbers of the colonies were then counted.

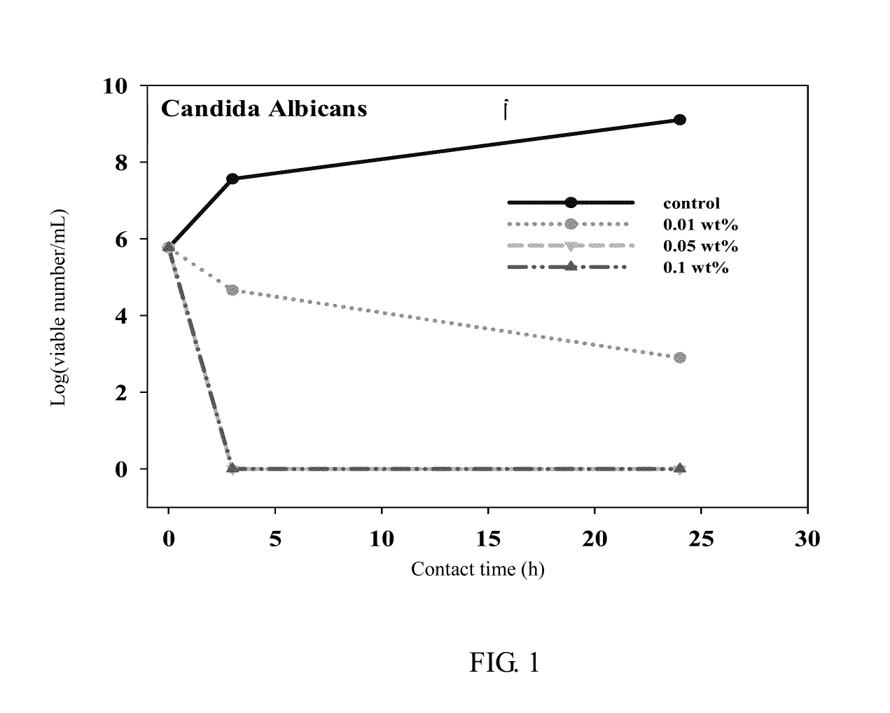

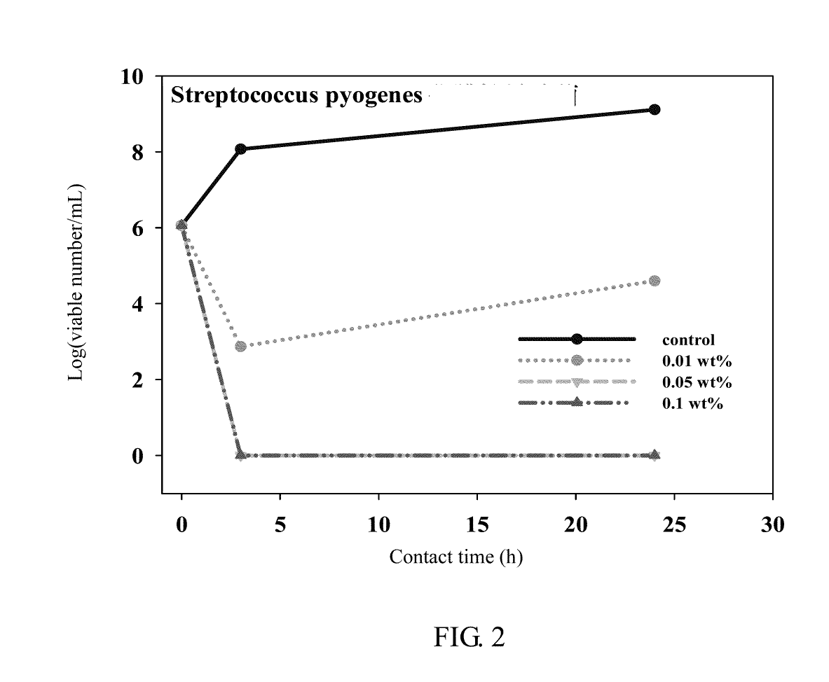

FIGS. 1˜5 show the results. After being cultured for 3 hours, the Candida albicans and the streptococcus pyogenes were completly inhibited in the media containing the AgNP/NSP composite (0.05 wt %). After being cultured for 24 hours, the Candida albicans and the streptococcus pyogenes in the media containing the AgNP/NSP composite (0.01 wt %) were partially inhibited. When comparred with the control group (no silver or other pharmaceuticals added), the effects of inhibiting bacteria were 100%. After contacting with the materials for 24 hours, staphylococcus aureus, proteus and pseudomonas aeruginosa can be completely inhibitted by the AgNP/NSP composites (0.01 wt %).

### B. In Vitro Cytotoxicity Tests of the AgNP/NSP Composites on Mammals

1. AgNP/NSP=7/93 (w/w)

The mammal CHO (Chinese hamster ovary) cells and Hs68 cells (human foreskin fibroblast) were used for evaluating the damage of the AgNP/NSP composite to cells. 3-(4,5)-dimethylthiahiazo (-z-yl)-3,5-di-phenyletra-zoliumromide (MTT) is a yellow pigment which can be reductively metabolized by succinate dehydrogenase in mitochondrial of the alive cells and generate blue or purple-blue water-insoluble formazan by reacting with cytochrome C. The maximun absorbance of formazan was at the wavelength 570 nm. In general, the production of formazan was proportioned to numbers of the alive cells, and thus the alive cells can be estimated from the OD (optical density). As the dead cells did not include succinate dehydrogenase, no reaction occurred after MTT was added.

In each incubating dish, 5×10⁴ cell/well of CHO cells and 5×10⁴ cell/well of Hs68 cells were planted. The incubator was then filled with 5% of CO₂ gas and the cells were incubated at 37° C. for 24 hours. Then water solutions of the AgNP/NSP composites (1, 0.75, 0.5, 0.25, 0.125 mg/ml) were respectively added into the dishes for incubating for 24 hours. Then the water solutions of MTT (10%) were added into the dishes for reacting with the AgNP/NSP composites and then the dishes were placed in incubator for 2 hours. Then the purple-blue crystals formed by alive cells were dissolved by DMSO (dimethy sulfoxide, in proper amounts) and OD values thereof were measured at wavelength 570 nm. By calculating cell proliferations (%), cytotoxicity of the AgNP/NSP composites can be estimated.

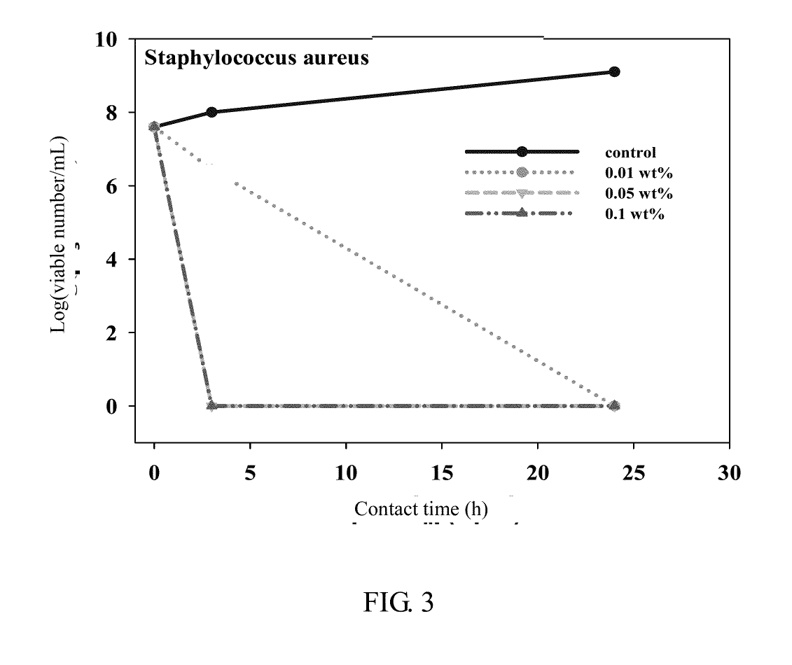

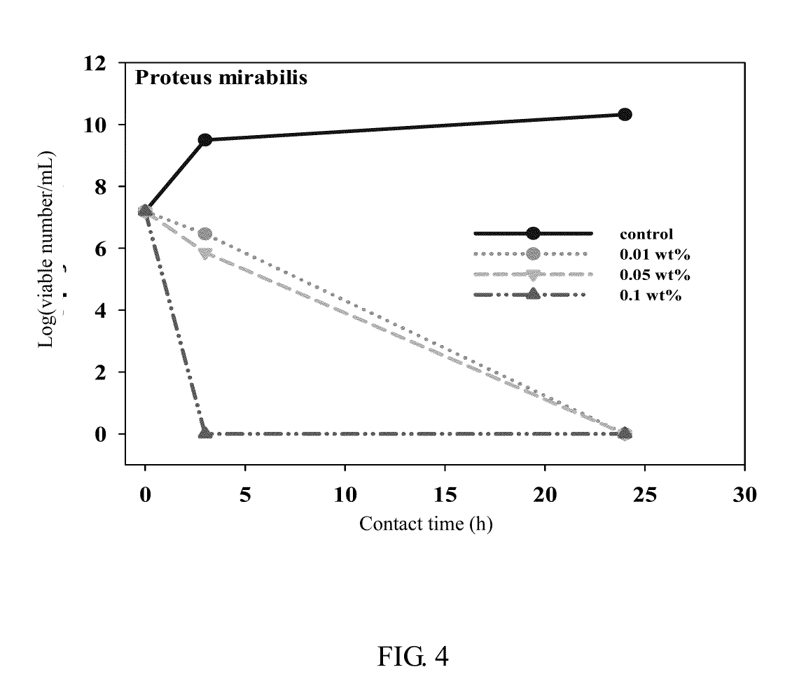

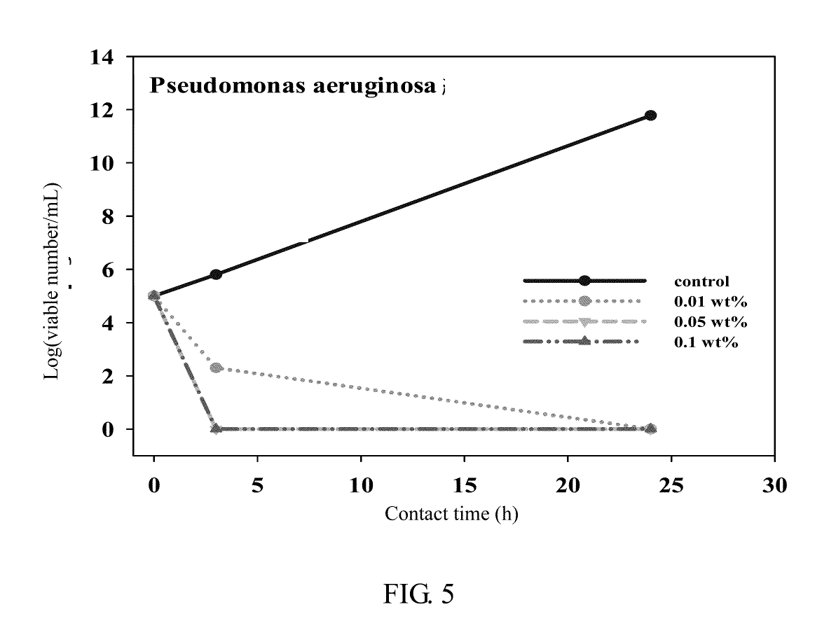

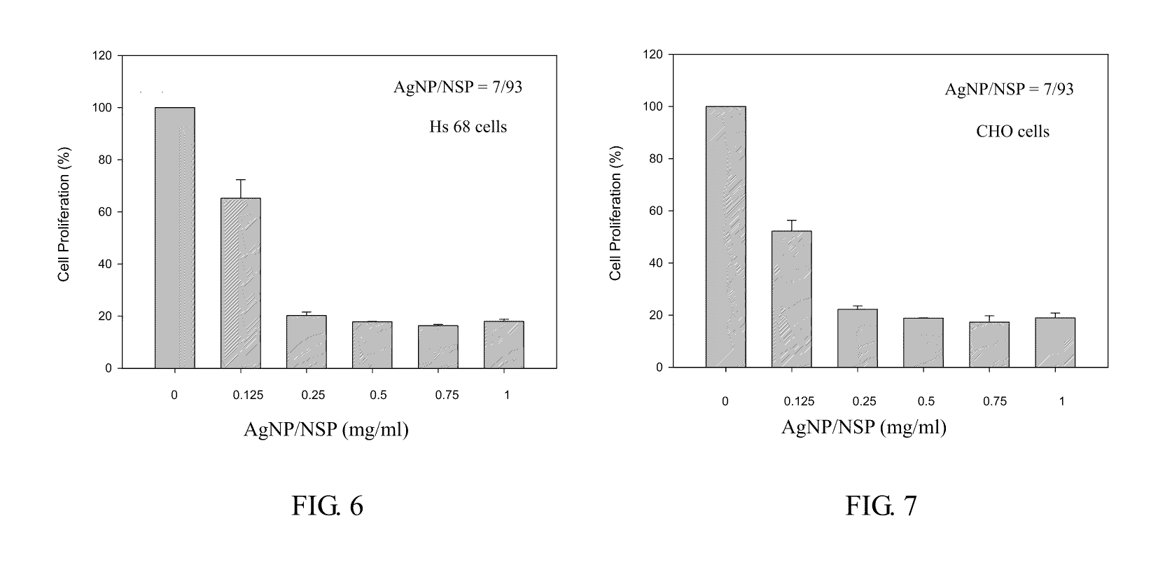

FIGS. 6 and 7 show the cell proliferations of Hs 68 cells and CHO cells, respectively. When the concentration of the AgNP/NSP composites was 0.25 mg/ml or higher, the cell proliferations were less than 30%. When the concentration was 0.125 mg/ml, the cell proliferations were 50-70%.

2. AgNP/NSP=7/93, 4/96, 1/99 (w/w)

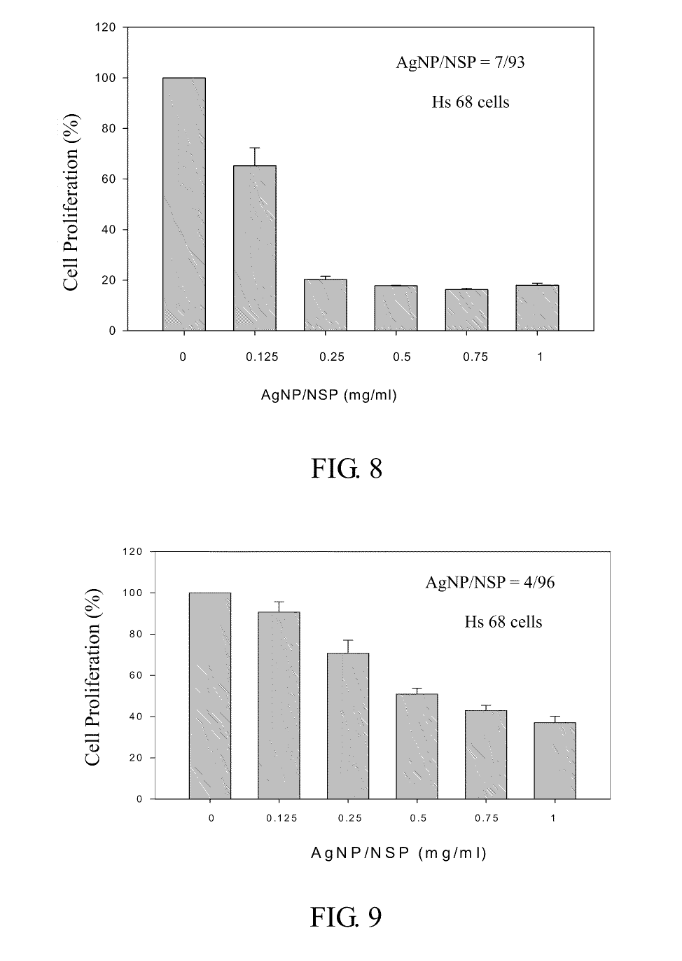

The procedures were the same as the above, except that the weight ratios of the AgNP/NSP composites were 7/93, 4/96, and 1/99. FIGS. 8-10 show the results. FIG. 8 was the same as FIG. 6.
1. When the Ag concentration was the same (17.5 ppm, or the concentration of AgNP/NSP=0.125 mg/ml), cell proliferations of the cells were about 20%, 70% and 80% (AgNP/NSP=7/93, 4/96 and 1/99). That is, in the same Ag concentration, toxicity decreased with increasing of clay.
2. IC50 was about 8.75 ppm, 35 ppm and 52.5 ppm (AgNP/NSP=7/93, 4/96 and 1/99). That is, cytotoxicity: 1/99<4/96<7/93.
3. When the weight ratio of AgNP/NSP was 1/99, toxicity was least. That is, clay can effectively decrease toxicity of silver.
4. Increasing of the death rates of cells in the media (AgNP/NSP=1/99) with concentrations was more moderate than those of the cells (AgNP/NSP=96/4, 93/7).

Accordingly, NSP did perform the effect in decreasing toxicity of silver.

### C. In Vitro Genotoxicity Tests on the Mammal Cells

Comet assay of the mammal cells is also known as single cell gel electrophoresis (SCGE). When DNA of cells was damaged, the damaged DNA will migrate from the nucleus in an electrophoresis field and form a tail. By measuring widths of the cell nuclei and distances of the tails, genotoxicity can be estimated.

In several incubating dishes, 5×10⁵ cell/well of CHO cells were added and then the dishes were placed in an incubator filling with 5% of CO₂ gas for incubation at 37° C. for 24 hours. Then water solutions of the AgNP/NSP composites (1, 0.75, 0.5, 0.25, 0.125 mg/ml) were added into the dishes and incubated in the incubator for 24 hours. Then the cells were isolated in a centrifuge at 1000 rpm for 5 minutes. The cells were then disrupted to release DNA from nuclei, and fixed on the two-layered agarose for SCGE at 13 volt for 20 minutes. The glasses were then dyed and observed under the fluorescent microscope.

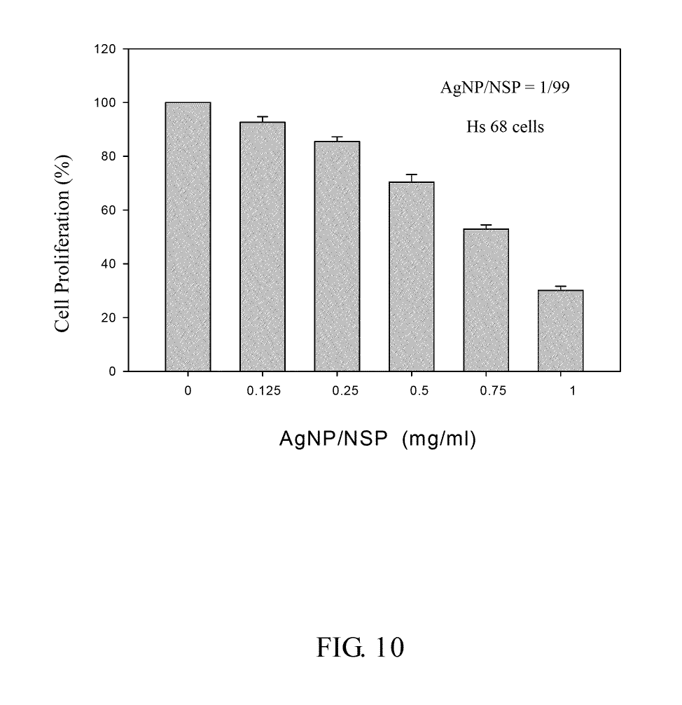

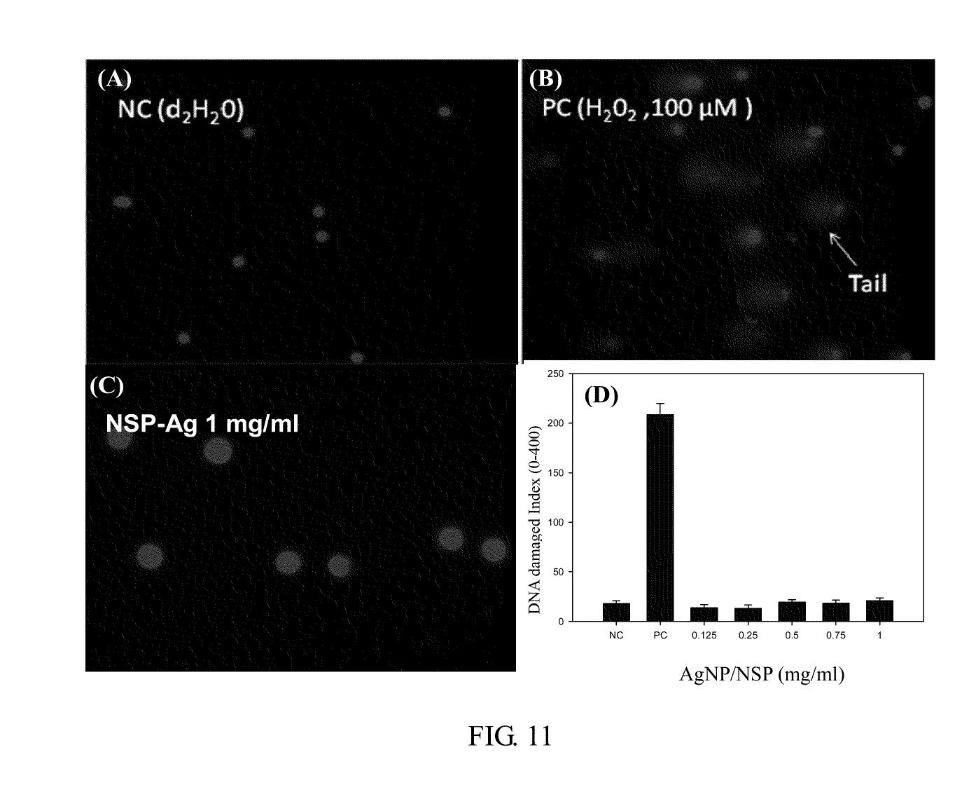

FIG. 11 showed the results, wherein (A) showed the undamaged DNA, (B) showed the damaged DNA having tails after H₂O₂ (100 μM) was added, (C) showed the undamaged DNA after AgNP/NSP (1 mg/ml) was added and (D) showed DNA damaged index. Compared to the negative control group (adding water) and the positive control group (adding H₂O₂), DNA of the cells of the tested groups would not be damaged by AgNP/NSP in high concentration (1 mg/ml).

### D. The Gene Mutation Assay for the Bacteria

When the salmonella mutation was irritated by mutagens, the wild colonies have the ability to assemble histidine by reversion of auxotrophic mutation. By testing selective media of lacking histidine, mutagen or carcinogen of chemicals can be determined. Each colony possessed different histidine operons. Colonies TA98, TA100, TA102, TA1535 and TA1537 showed characteristic of ΔuvrB and defect in DNA excision repair, so that the damaged DNA might be observed. Colonies TA97, TA98, TA100, TA102 and TA1535 possess characteristic of rfa, i.e., partial defect of the lipopolysaccharide barrier on cell walls of colonies, and thus osmosis of chemical molecules into bacteria would increased. Colonies TA97, TA98, TA100 and TA102 were induced with pkM101 plasmid and could trend to be incorrectly repaired. Since the damaged DNA were not easily repaired and would be more sensitive.

On the first day, in an incubator filling with 5% of CO₂, different salmonella (TA98, TA100, TA102, TA1535 and TA1537) were incubated in NB liquid media at 37° C. On the second day, bacteria histidine and AgNP/NSP solution were added into sterilized soft agar, then placed in solid nutrient plates for 2 or 3 days and colonies were counted.

ATTACHMENTs 1 and 2 showed the results. ATTACHMENT 1 showed the gene mutation assay of the bacteria without enzyme metabolism (−S9). ATTACHMENT 2 showed the gene mutation assay of the bacteria with enzyme metabolism (+S9). The AgNP/NSP could inhibit salmonella in 1 mg/ml and had no genotoxicity in 0.75 mg/ml.

### E. Treatments of Scalds/Burns of Mice

Rare mice were anesthetized by intra-peritoneal injecting chloral hydrate (3.7%, 0.15˜0.2 ml) and disinfected abdomen with alcohol. A metal plate was heated to 80° C. and then attached to abdomen of the bare mice for 30 minutes. Area of each wound was 1.5×1.5 cm². Then the wounds were scraped with an aseptic scalpel to expose dermis, which was the test model of first- or second-degree scalds/burns. For the first and second groups, germfree gauze (each 2 cm², spread with bacteria 100 μl) was pasted on wounds. For the third and forth groups, germfree gauze (each 2 cm², spread with bacteria 100 μl and silver sulfadiazine 200 ul) was pasted on wounds. For the fifth and sixth groups, germfree gauze (each 2 cm², spread with bacteria 100 μl and AgNP/NSP 200 ul) was pasted on wounds. On the sixth day, antibacterial effects was evaluated by observing the skinning over of the wounds with rare eyes.

As a result, silver sulfadiazine used in the third and forth groups (AgNP/NSP) performed good effect in inhibitting E. coli strain J53 pMG101, wherein the third group (1 wt % AgNP/NSP) was the most siganifacant. On the sixth day, eschar still adhered to the wound, that is, the new dermis did not grow well.

For AgNP/NSP, effects of inhibitting J53PMG 101 could be also observed through the first to third days. Therefore, noninvasive damage was prevented and infection was limited on epidermis. On the sixth day, the fifth group (1 wt % AgNP/NSP) signifacantly skined over and eschar sloughed off. The neovessels under epidermis were identifable and the healed skin was very similar to the infective skin. That is, AgNP/NSP (1 wt %) could show signifacant antibacterial effect.

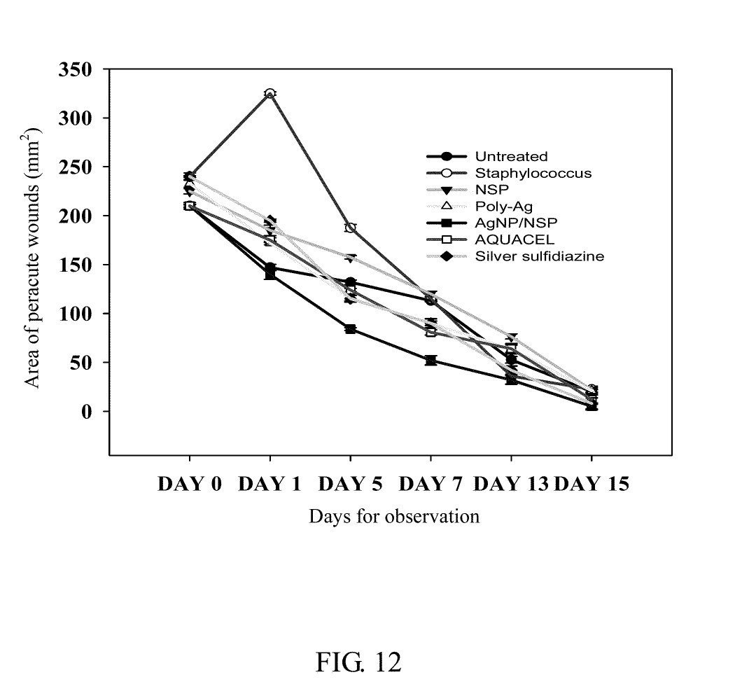

FIG. 12 showed areas of the wounds treated in differnt manners on the 2nd, 4th and 7th days. As shown in the figure, the wounds treated with Aquacel, silver sulfadiazine and AgNP/NSP respectively had areas 130 mm², 112 mm² and 98 mm² That is, AgNP/NSP could perform better effect in skinning over than Aquacel and silver sulfadiazine.

### F. Evaluation on Peracute and Chronic Wounds

To widely apply AgNP/NSP to animals, two models were respectively built by peracute wounds and chronic wounds.

The peracute wounds were scalds/burns caused by attaching a metal plate (1.5×1.5 cm², 180° C.) on backs of bare mice for 15 seconds. Then differnt materials were used to treat the wounds and areas and statuses thereof were observed.

The chronic wounds (each 1.5×1.5 cm²) were formed by cutting skin of backs of mice with a sterilized scalpel. Then differnt materials were used to treat the wounds and areas and statuses thereof were observed.

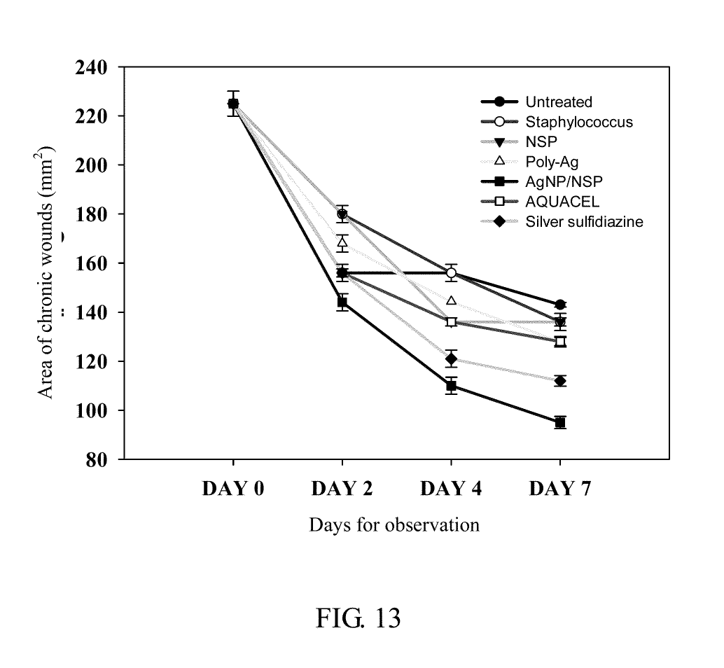

FIG. 13 showed areas of the wounds treated in differnt manners on the 1st, 5th, 7th, 13th and 15th days. On the first day, AgNP/NSP performed effect in inhibiting bacteria and the area of the wound maintained the smallest compared with silver sulfadiazine and Aquacel. That is, AgNP/NSP also had good effect in skinning over of chronic wounds.

ATTACHMENT 1

| | S. Typhimurium strain | | | | |
|---|---|---|---|---|---|
| AgNP/NSP | (− S9) (mg/ml · colony) | | | | |
| (mg/plate) | TA98 | TA100 | TA102 | TA1535 | TA1537 |
| NC | 47 ± 4 | 227 ± 7 | 247 ± 8 | 12 ± 2 | 11 ± 4 |
| 0.125 | 52 ± 4 | 237 ± 11 | 255 ± 6 | 9 ± 3 | 8 ± 1 |
| 0.250 | 48 ± 2 | 220 ± 19 | 241 ± 4 | 15 ± 5 | 9 ± 3 |
| 0.500 | 37 ± 4 | 183 ± 4 | 239 ± 6 | 11 ± 2 | 10 ± 2 |
| 0.750 | 36 ± 3 | 102 ± 10 | 242 ± 3 | 7 ± 3 | 9 ± 2 |
| 1.000 | 31 ± 2 | 89 ± 15 | 221 ± 3 | 4 ± 1 | 6 ± 1 |
| PC | 483 ± 13 | 657 ± 22 | 2089 ± 18 | 149 ± 9 | 152 ± 7 |

ATTACHMENT 2

| | S. Typhimurium strain | | | | |
|---|---|---|---|---|---|
| AgNP/NSP | (+ S9) (mg/ml · colony) | | | | |
| (mg/plate) | TA98 | TA100 | TA102 | TA1535 | TA1537 |
| NC | 39 ± 3 | 169 ± 5 | 207 ± 10 | 21 ± 2 | 11 ± 2 |
| 0.15 | 42 ± 5 | 147 ± 11 | 224 ± 4 | 24 ± 2 | 10 ± 1 |
| 0.25 | 43 ± 3 | 158 ± 6 | 203 ± 7 | 17 ± 3 | 6 ± 1 |
| 0.50 | 35 ± 4 | 154 ± 4 | 197 ± 4 | 19 ± 1 | 8 ± 1 |
| 0.75 | 29 ± 2 | 142 ± 5 | 191 ± 5 | 16 ± 1 | 5 ± 1 |
| 1.00 | 28 ± 3 | 148 ± 7 | 184 ± 6 | 15 ± 2 | 5 ± 2 |
| PC | 324 ± 6 | 537 ± 12 | 2294 ± 17 | 103 ± 9 | 75 ± 5 |

What is claimed is:

1. A composite of metallic nanoparticles and inorganic clay for treating a wound, the composite having a weight ratio of the metallic nanoparticles to the inorganic clay ranging from 0.1/99.9 to 6.0/94.0, and a size from 5 nm to 100 nm; wherein the inorganic clay has an aspect ratio from 10 to 100,000 and serves as a carrier of the metallic nanoparticles.

2. The composite of metallic nanoparticles and inorganic clay of claim 1, wherein the weight ratio of the metallic nanoparticles to the inorganic clay ranges from 0.5/99.5 to 3/97.

3. The composite of metallic nanoparticles and inorganic clay of claim 1, wherein the weight ratio of the metallic nanoparticles to the inorganic clay ranges from 0.5/99.5 to 2/98.

4. The composite of metallic nanoparticles and inorganic clay of claim 1, wherein the metallic nanoparticles are gold, silver, copper or iron.

5. The composite of metallic nanoparticles and inorganic clay of claim 1, wherein the inorganic clay is nanosilicate platelets, montmorillonite (MMT), bentonite, laponite, synthetic mica, kaolinite, talc, attapulgite clay, vermiculite or layered double hydroxides (LDH).

6. The composite of metallic nanoparticles and inorganic clay of claim 1, wherein the ratio of the ionic equivalent of the metal particles to the cation exchange equivalent of the inorganic layered clay is 0.1 to 200.

7. A method for producing a composite of metallic nanoparticles and inorganic clay, comprising a step of mixing and reacting metallic particles, layered inorganic clay and a reducing agent to generate the composite having a size of 5 to 100 nm,
wherein the weight ratio of the metallic nanoparticles to the layered inorganic clay ranges from 0.1/99.9 to 6.0/94.0;
the layered inorganic clay has an aspect ranging from 10 to 100,000 and serves as carriers of the metallic nanoparticles to disperse the metallic nanoparticles on a nano scale.

8. The method of claim 7, wherein the weight ratio of the metallic nanoparticles to the layered inorganic clay ranges from 0.5/99.5 to 3/97.

9. The method of claim 7, wherein the weight ratio of the metallic nanoparticles to the layered inorganic clay ranges from 0.5/99.5 to 2/98.

10. The method of claim 7, wherein the metallic particles are gold, silver, copper or iron.

11. The method of claim 7, wherein the layered inorganic clay is nanosilicate platelets, montmorillonite (MMT), bentonite, laponite, synthetic mica, kaolinite, talc, attapulgite clay, vermiculite or layered double hydroxides (LDH).

12. The method of claim 7, wherein the reducing agent is methanol, ethanol, propanol, butanol, formaldehyde, ethylene glycol, propylene glycol, butanediol, glycerine, PVA (polyvinyl alcohol), PEG (polyethylene glycol), PPG (polypropylene glycol), dodecanol or sodium borohydride (NaBH₄).
